(19) United States
(12) Patent Application Publication
(10) Pub. No.: US 2017/0348322 A1
(43) Pub. Date: Dec. 7, 2017

(54) BK Channels as Non-Genomic Estrogen Targets for Overactive Bladder

(71) Applicant: University of South Carolina, Columbia, SC (US)

(72) Inventor: Georgi V. Petkov, Columbia, SC (US)

(73) Assignee: University of South Carolina, Columbia, SC (US)

(21) Appl. No.: 15/614,725

(22) Filed: Jun. 6, 2017

Related U.S. Application Data
(60) Provisional application No. 62/346,601, filed on Jun. 7, 2016.

(51) Int. Cl. A61K 31/565; C12N 5/071; A61K 9/00; G01N 33/68; A61K 9/20; C12N 5/077; A61K 45/00; A61K 31/00

(52) U.S. Cl. CPC A61K 31/565; A61K 9/2013; C12N 5/0685; C12N 5/0661; A61K 9/0024; G01N 33/6872; A61K 31/00; A61K 45/00

(57) ABSTRACT

Using BK Channels as non-genomic estrogen targets in order to alleviate overactive bladder.

This disclosure was developed with the use of research funds from the National Institute of Health (“NIH”) pursuant to Grant Number NIH R01 DK084284.

### BACKGROUND OF THE INVENTION

### 1) Field of the Invention

The present disclosure relates to the use of BK Channels as non-genomic estrogen targets in order to alleviate overactive bladder.

### 2) Description of Related Art

The functions of the urinary bladder, which are to store and periodically release urine, are facilitated by the contraction and relaxation of urinary bladder smooth muscle (UBSM). Overactive bladder (OAB), a highly prevalent chronic health condition in the United States, is often associated with increased UBSM contractility. Many forms of OAB have been linked directly to UBSM dysfunction. Therefore, novel therapeutic modalities for OAB, targeting UBSM directly, are urgently needed.

OAB is the most common form of lower urinary tract dysfunction (LUTD). OAB, often associated with detrusor overactivity (DO), affects 17% of the population and its prevalence increases with age. OAB remains a poorly understood condition that presents a significant medical challenge. Although some therapeutic options are available for the treatment of OAB, currently there is no universally effective OAB therapy.

While antimuscarinics are the primary pharmacological treatment for OAB, the clinical use of these agents provides limited efficacy and undesirable side effects. Antimuscarinic drugs have been used for the treatment of more or less specified gastrointestinal diseases or complaints for many centuries, first in herbal preparations (including belladonna), and in modern times as synthetic tertiary or quaternary compounds, with atropine being a pharmacological standard. Conventional antimuscarinics act unselectively on receptors in heart, smooth muscle and exocrine glands.

The long-term effectiveness of newer therapies—such as mirabegron, a selective β3-adrenoceptor agonist and botulinum toxin—remain uncertain, and in some cases their use presents safety concerns. The lack of safe and universally effective OAB treatments continues to spur the scientific community to seek novel therapeutic approaches to control OAB.

Complex and coordinated regulatory mechanisms involving hormones, neurotransmitters, receptors, and ion channels regulate the physiology and pathophysiology of detrusor smooth muscle (DSM). Increasing evidence suggests that sex hormones, specifically 17β-estradiol, have a critical role in the control of DSM function. Yet, the molecular and cellular mechanisms underlying 17β-estradiol-mediated regulation of human DSM physiology have not been fully elucidated. Estrogen receptors are expressed in the bladder, urethral, vaginal, and pelvic floor smooth muscles; however, controversy in the literature concerning the role of estrogens in urinary bladder function exist. For example, estrogens have been shown to stimulate DSM contractility in rabbits, while DSM relaxation was reported in pigs, rats, and guinea pigs.

Current research has suggested a potential role for estrogen replacement therapies in the treatment of bladder disorders including overactive bladder (OAB) and urinary incontinence. Some studies have reported positive results for estrogen therapies in mitigating symptoms of OAB in postmenopausal women, while other studies reported the contrary. Large epidemiological studies investigating the use of systemic hormone replacement therapy in the prevention of cardiovascular disease and osteoporosis revealed that these therapies may increase the risk for development of urinary incontinence. However, more recent meta-analysis supported the use of local, but not systemic, estrogen therapies for the treatment of urge urinary incontinence and OAB. Therefore, an improved understanding of the roles of estrogens in bladder function may lead to greater insight concerning the efficacy of estrogen replacement therapies for OAB treatment.

Accordingly, it is an object of the present invention to provide effective OAB therapies.

### SUMMARY OF THE INVENTION

In one embodiment, the current disclosure provides a method of regulating detrusor smooth muscle function. The method includes administering a compound comprising an estrogen hormone, regulating excitation-contraction coupling in detrusor smooth muscle via BK channel activation, increasing depolarization of detrusor smooth muscle cells, and reducing EFS-induced contractions at physiologically-relevant stimulation frequencies. In a further embodiment, the estrogen hormone comprises 17β-estradiol. In a still further embodiment, a potassium channel blocker is administered to counteract stimulatory effects of the estrogen hormone. In a yet further embodiment, the estrogen hormone increases BK channel NPo. Still further, BK channel amplitude remains substantially unaffected. In a further embodiment, regulating detrusor smooth muscle function is achieved independent of genomic estrogen receptors. Still yet further, regulating detrusor smooth muscle function is achieved by direct modulation of the smooth muscle-specific regulatory BK channel β₁-subunit. In a further embodiment, the estrogen hormone hyperpolarizes a resting membrane potential in a BK-channel-dependent manner. Still further yet, the physiologically-relevant stimulation frequencies are from 0.5 to 50.0 Hz.

In an alternative embodiment, a hormonal therapeutic intervention is disclosed. The intervention includes administering a therapeutic agent comprising an estrogen hormone, regulating excitation-contraction coupling in detrusor smooth muscle via BK channel activation, and controlling neurogenically mediated detrusor dysfunction. In a further embodiment, the estrogen hormone comprises 17β-estradiol. In a still further embodiment, 17β-estradiol rapidly activates the BK channels by directly targeting the BK channel rather than targeting intracellular signaling pathways. In a still yet further embodiment, a potassium channel blocker is administered to counteract stimulatory effects of the estrogen hormone. In still further embodiment, the estrogen hormone increases BK channel NPo. In a yet further embodiment, BK channel amplitude remains substantially unaffected. Still further, regulating detrusor smooth muscle function is achieved independent of genomic estrogen receptors. Yet further still, regulating detrusor smooth muscle function is achieved by direct modulation of a smooth muscle-specific regulatory BK channel β₁-subunit. In a further embodiment, the estrogen hormone hyperpolarizes a resting membrane potential in a BK-channel-dependent manner.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows a proposed mechanism of estrogen effects on BK channels in DSM cells.

FIG. 2 illustrates that 17β-Estradiol increases the depolarization-induced whole cell outward currents in freshly-isolated DSM cells.

FIG. 3 shows that 17β-Estradiol increases transient BK current (TBKC) activity in freshly-isolated DSM cells.

FIG. 4 illustrates that 17β-Estradiol increases the single BK channel open probability (NPo) of human DSM excised membrane patches.

FIG. 5 shows that 17β-Estradiol hyperpolarizes the resting membrane potential (RMP) in DSM cells.

FIG. 6 shows that 17β-Estradiol inhibits spontaneous phasic contractions of human DSM isolated strips.

FIG. 7 illustrates that 17β-Estradiol inhibits the nerve-evoked contractions of human DSM isolated strips.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Estrogens have an important modulatory role in regulating detrusor smooth muscle (DSM) function. However, the underlying molecular and cellular mechanisms by which estrogens control human DSM excitability and contractility are not well known. In the current disclosure, human DSM specimens were used from open bladder surgeries on 27 patients to elucidate the mechanism by which 17β-estradiol regulates large conductance voltage- and Ca2+-activated K+ (BK) channels, the most prominent K+ channels in human DSM.

We employed the perforated patch-clamp technique on freshly-isolated DSM cells and smooth muscle tension recordings on DSM isolated strips to investigate the mechanism by which 17β-estradiol activates BK channels. 17β-Estradiol (100 nM) rapidly increased depolarization-induced whole cell K+ currents in DSM cells. This 17β-estradiol-induced potentiation of the whole cell BK currents was completely abolished by the selective BK channel inhibitor paxilline (1 μM), clearly indicating that 17β-estradiol specifically activates BK channels. 17β-Estradiol also increased the frequency of ryanodine receptor-mediated transient BK currents. Single BK channel recordings showed that 17β-estradiol (100 nM) significantly increased the BK channel open probability of inside-out excised membrane patches, revealing that 17β-estradiol activates BK channels directly. 17β-Estradiol reduced spontaneous phasic contractions of human DSM isolated strips in a concentration-dependent manner (100 nM-1 μM). The inhibitory effects of 17β-estradiol on DSM spontaneous phasic contractions were blocked by paxilline (1 μM). 17β-Estradiol (100 nM) also reduced nerve-evoked contractions of human DSM strips. Collectively, our results reveal that 17β-estradiol plays a critical role in regulating human DSM function through a direct non-genomic activation of BK channels.

Ion channels are particularly important targets for mediating the effects of estrogens. For example, 17β-estradiol has been shown to interact with K+ channels, including voltage-gated K+ and TASK channels, a member of the two-pore domain K+ channel family. Furthermore, 17β-estradiol has been reported to activate the large conductance voltage- and Ca2+-activated K+ (BK) channels in some smooth muscles including cultured human coronary artery smooth muscle cells and colonic myocytes. As BK channels are fundamental regulators of DSM function under normal and OAB conditions, when the channel expression is altered, pharmacological modulation of their activity could potentially be an effective approach to treat forms of lower urinary tract dysfunction, such as OAB.

A recent study revealed that 17β-estradiol activates BK channels in guinea pig DSM cells through direct non-genomic mechanisms. However, the functional interaction between 17β-estradiol and BK channels in human DSM is unknown. Significant species-related differences in animal versus human DSM excitability and contractility have been well documented. Moreover, studies on human DSM cells and tissues are of critical importance to validate the findings obtained from animal models.

In the current disclosure, we used a multidisciplinary experimental approach including amphotericin B-perforated whole cell patch-clamp electrophysiology, single BK channel recordings, and isometric DSM tension recordings to test the hypothesis that 17β-estradiol decreases the excitability and contractility of human DSM via mechanisms involving direct BK channel activation.

Materials and Methods

Human DSM specimen collection: The study forming the basis for the current disclosure was conducted according to protocol Pro00045232, reviewed and approved by the Medical University of South Carolina Institutional Review Board. Human bladder specimens were obtained from 27 patients (20 male and 7 female, average age of 69.0±2.1 years, 25 Caucasians and 2 Hispanic), who did not have a preoperative history of OAB. The DSM tissue samples were obtained during open bladder surgeries performed for a variety of indications such as bladder cancer, including radical cystectomy for urothelial carcinoma, and adenocarcinoma. In such cases, the collected DSM tissue was remote from the site of tumor. DSM strips were dissected from the bladder specimens, and after removing the urothelial and lamina propria layers, the strips were used for single DSM cell isolation and isometric DSM tension recordings.

Isometric DSM tension recordings: Isometric DSM tension recordings of human DSM isolated strips (˜7-8 mm long and ˜4-5 mm wide) were conducted as previously described (Hristov K L, Chen M, Kellett W F, Rovner E S, and Petkov G V. Large-conductance voltage- and Ca2+-activated K+ channels regulate human detrusor smooth muscle function. Am J Physiol Cell Physiol 301: C903-912, 2011; Hristov K L, Parajuli S P, Soder R P, Cheng Q, Rovner E S, and Petkov G V. Suppression of human detrusor smooth muscle excitability and contractility via pharmacological activation of large conductance Ca2+-activated K+ channels. Am J Physiol Cell Physiol 302: C1632-1641, 2012).

DSM single cell isolation: Human single DSM cells were enzymatically isolated as previously described. (Hristov K L, Chen M, Kellett W F, Rovner E S, and Petkov G V. Large-conductance voltage- and Ca2+-activated K+ channels regulate human detrusor smooth muscle function. Am J Physiol Cell Physiol 301: C903-912, 2011; Hristov K L, Parajuli S P, Soder R P, Cheng Q, Rovner E S, and Petkov G V. Suppression of human detrusor smooth muscle excitability and contractility via pharmacological activation of large conductance Ca2+-activated K+ channels. Am J Physiol Cell Physiol 302: C1632-1641, 2012; Parajuli S P, Hristov K L, Cheng Q P, Malysz J, Rovner E S, and Petkov G V. Functional link between muscarinic receptors and large-conductance Ca2+-activated K+ channels in freshly isolated human detrusor smooth muscle cells. Pflugers Arch 467: 665-675, 2015.)

Patch-clamp electrophysiological experiments: Patch-clamp experiments were performed as previously described. (Hristov K L, Chen M, Kellett W F, Rovner E S, and Petkov G V. Large-conductance voltage- and Ca2+-activated K+ channels regulate human detrusor smooth muscle function. Am J Physiol Cell Physiol 301: C903-912, 2011; Hristov K L, Parajuli S P, Soder R P, Cheng Q, Rovner E S, and Petkov G V. Suppression of human detrusor smooth muscle excitability and contractility via pharmacological activation of large conductance Ca2+-activated K+ channels. Am J Physiol Cell Physiol 302: C1632-1641, 2012; Malysz J, Rovner E S, and Petkov G V. Single-channel biophysical and pharmacological characterizations of native human large-conductance calcium-activated potassium channels in freshly isolated detrusor smooth muscle cells. Pflugers Arch 465: 965-975, 2013; Parajuli S P, Hristov K L, Cheng Q P, Malysz J, Rovner E S, and Petkov G V. Functional link between muscarinic receptors and large-conductance Ca2+-activated K+ channels in freshly isolated human detrusor smooth muscle cells. Pflugers Arch 467: 665-675, 2015.)

The current disclosure applied the amphotericin B-perforated whole cell patch-clamp technique to record transient BK currents (TBKCs), depolarization-induced steady-state whole cell BK currents, and the resting membrane potential (RMP) of human freshly-isolated DSM cells. TBKCs in DSM cells were recorded at the holding potential of −20 mV. Depolarization-induced steady-state whole cell BK currents were elicited by holding the DSM cells at −70 mV and then brief, 200 ms depolarization steps were applied from −40 mV to +80 mV in increments of 20 mV. Single BK channel activity was recorded from inside-out excised membrane patches using symmetrical K⁺ (140 mM) solutions, at a holding potential of −60 mV (Vₕ=−60 mV). The electrochemical driving force for K⁺ in these experimental conditions was +60 mV (V_{DV}=60 mV). Paxilline (1 μM), a selective BK channel inhibitor was used to dissect their functional role in mediating 17β-estradiol induced effects on DSM function. All patch-clamp experiments were conducted at room temperature (22-23° C.).

Solutions and drugs: Ca²⁺-free dissection solution contained (in mM): 80 monosodium glutamate, 55 NaCl, 6 KCl, 10 glucose, 10 HEPES,2 MgCl₂, and the pH was adjusted to 7.3 with NaOH. For the functional studies on human DSM contractility, the physiological saline solution had the following composition (in mM): 119 NaCl, 4.7 KCl, 24 NaHCO₃, 1.2 KH₂PO₄, 2.5 CaCl₂, 1.2 MgSO₄, and 11 D-glucose, aerated with 95% O₂-5% CO₂ (pH 7.4). Extracellular (bath) solution used for the perforated whole cell patch-clamp experiments contained (in mM): 134 NaCl, 6 KCl, 1 MgCl₂, 2 CaCl₂, 10 glucose, 10 HEPES, and pH was adjusted to 7.4 with NaOH. The patch-pipette solution for the perforated patch-clamp experiments contained (in mM): 110 potassium aspartate, 30 KCl, 10 NaCl, 1 MgCl₂, 10 HEPES, 0.05 EGTA, and pH was adjusted to 7.2 with NaOH. Amphotericin-B stock solution was prepared daily in dimethyl sulfoxide (DMSO) and was added to the pipette solution (200 μg/ml) before the experiment and was replaced every 1-2 h. Extracellular and patch-pipette solutions for single BK channel recordings contained (in mM): 140 KCl, 1.08 MgCl₂, 5 EGTA, 10 HEPES, and 3.16 CaCl₂, adjusted to pH 7.2 with NaOH (Ca²⁺ free concentration was calculated ˜300 nM with WEBMAXC Standard, http://www.stanford.edu/˜cpatton/webmaxcS.htm, Chris Patton). 17β-Estradiol and paxilline were applied by replacement of the extracellular solution via superfusion. 17β-Estradiol and paxilline were purchased from Sigma-Aldrich and were dissolved in DMSO. The final concentration of DMSO in the bath solution did not exceed 0.02%.

Data analysis and statistics: Clampfit 10.3 (Molecular Device, Union City, Calif.) and Minianalysis software (Synaptosoft, Inc., NJ) were used to analyze the data. Mean values of the last 50 ms pulse of 200 ms depolarization step of at least 5 average files in the absence (control) and in the presence of 17β-estradiol were analyzed to evaluate the effects of 17β-estradiol on steady-state whole cell BK currents. Only DSM cells with stable internal time controls of at least 8-10 min before application of 17β-estradiol were used in this study. Five minutes of at least an 8-10 min stable patch-clamp recording prior to application of 17β-estradiol were analyzed for control data, and the last 5 min of continuous recordings of 10-15 min after application of 17β-estradiol were analyzed to evaluate the effects of 17β-estradiol on RMP or TBKCs. The threshold of TBKCs was set at 9 pA. The values for single BK channel open probability (NPo) were obtained using the built-in algorithm in Clampfit, which calculates as NPo=(To)/(To+Tc), where To and Tc correspond, respectively, to total open time and closed time during the recording interval. Human DSM contractions were analyzed with MiniAnalisis software (Synaptosoft, Inc., NJ). The control values of each individual DSM strip were normalized and represented as 100%. GraphPad Prism 4.03 (GraphPad Software, Inc., La Jolla, Calif.) and CorelDRAW Graphics Suite X3 (Corel Co., Mountain View, Calif.) were used for the statistical analyses and data presentation. The data are expressed as mean±SEM for the “n” (the number of DSM cells or strips) isolated from “N” (the number of patients). Statistical analyses were performed using the paired Student's t test or ANOVA with Bonferroni's post-hoc test. A P value <0.05 was considered statistically significant.

Results

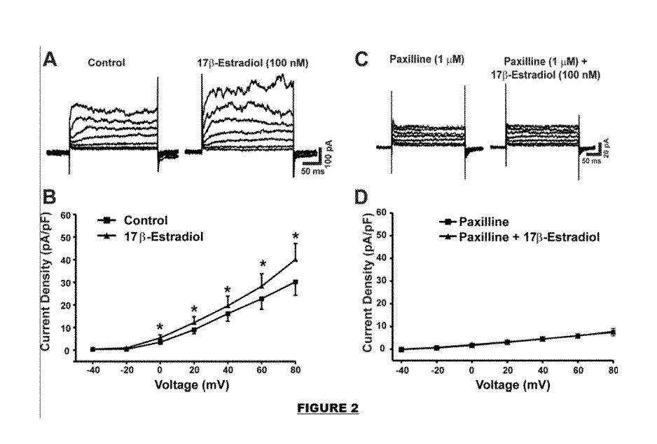

17β-Estradiol increases depolarization-induced steady-state whole cell outward BK currents in freshly-isolated human DSM cells. First, the current disclosure investigated the regulatory role of 17β-estradiol on human DSM cell excitability. The current disclosure studied the effects of 17β-estradiol on depolarization-induced steady-state whole cell outward BK currents by using amphotericin-B perforated patch-clamp and freshly-isolated DSM cells. DSM cells used in the present study had an average capacitance of 19.2±1.2 pF (n=43, N=22). Whole cell outward K⁺ currents were increased gradually in response to voltage-step depolarization from −40 mV to +80 mV as shown in FIG. 2. FIG. 2A shows representative original recordings illustrating the depolarization-induced whole cell outward currents in the absence (control) and in the presence of 17β-estradiol (100 nM); FIG. 2B shows the current-voltage relationship curve summarizes the stimulatory effects of 17β-estradiol on the whole cell outward currents (n=12, N=8; *P<0.05); FIG. 2C shows representative original recordings illustrating the depolarization-induced whole cell outward currents in the presence of paxilline (1 μM) alone and in the presence of both paxilline (1 μM) and 17β-estradiol (100 nM). D) The current-voltage relationship curve summarizes the lack of stimulatory effects of 17β-estradiol on the whole cell outward currents in the presence of 1 μM paxilline (n=6, N=6; P>0.05), FIG. 2A. 17β-Estradiol (100 nM) significantly increased the whole cell K⁺ current density in DSM cells, FIG. 2. At the highest recording voltage of +80 mV, the whole cell outward K⁺ currents were 33.9±6.5 pA/pF and 44.5±7.8 pA/pF in the absence and in the presence of 17β-estradiol, respectively (n=12, N=8; P<0.05; FIG. 2A-2B).

The effects of 17β-estradiol on whole cell outward K⁺ currents were completely abolished by the selective BK channel inhibitor paxilline (1 μM). At +80 mV, the BK current amplitudes were 6.1±1.7 pA/pF and 6.8±1.8 pA/pF in the presence of 1 μM paxilline alone and in the presence of both paxilline (1 μM) and 17β-estradiol (100 nM), respectively (n=6, N=6; P>0.05; FIG. 2C-D). The lack of 17β-estradiol stimulatory effects on whole cell outward K⁺ currents in the presence of paxilline suggests that the potentiating effects of 17β-estradiol involve exclusively BK channel activation.

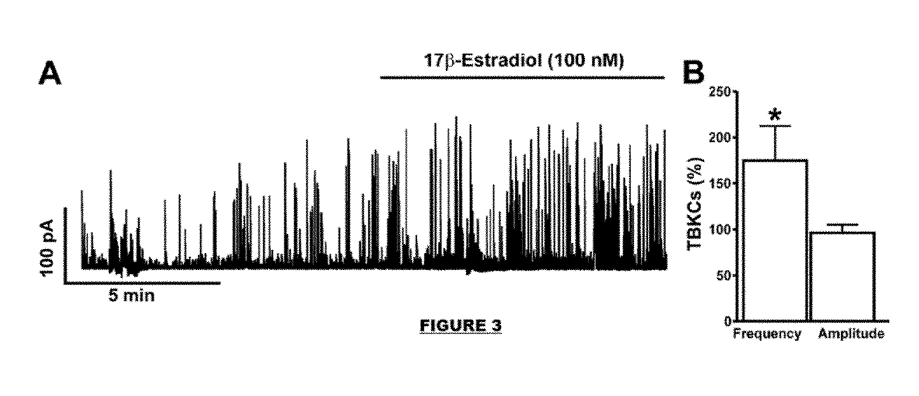

17β-Estradiol increases TBKC activity in freshly-isolated DSM cells. DSM cells generate TBKCs, caused by localized Ca²⁺ release events from the sarcoplasmic reticulum, known as Ca²⁺ sparks, which leads to subsequent BK channel activation ENREF 27. At a holding potential of −20 mV, 17β-estradiol (100 nM) increased the frequency of TBKCs by 74.6±37.8% without significantly altering the average TBKC amplitude (n=8, N=5; P<0.05; FIG. 3). FIG. 3A shows a representative original recording illustrating the stimulatory effect of 100 nM 17β-estradiol on the frequency of TBKCs in human DSM single cell. FIG. 3B shows summary data illustrating the stimulatory effects of 17β-estradiol (100 nM) on the frequency of TBKCs (n=8, N=5; *P<0.05). The data were normalized to control values (prior to 17β-estradiol addition) taken as 100% and were presented as percentages (%). TBKCs were recorded at a holding potential of −20 mV. The results suggest that activation of BK channels with 17β-estradiol enhances TBKCs frequency in DSM cells.

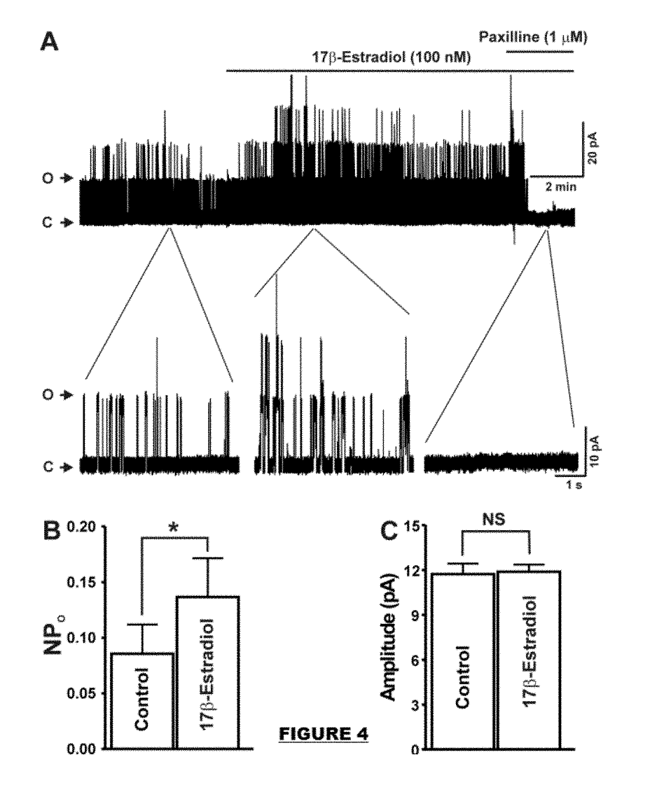

17β-Estradiol increases single BK channel open probability in the excised membrane patches of human DSM cells. To further investigate the precise cellular mechanism by which 17β-estradiol activates BK channels in DSM cells, the current disclosure conducted a series of experiments on single BK channel activity by using the inside-out excised-patch configuration of the patch-clamp technique. 17β-Estradiol (100 nM) significantly increased the single BK channel NPo from 0.086±0.030 to 0.137±0.030 (n=9, N=7, P<0.05; FIG. 4). FIG. 4A shows a representative original recording from excised membrane patch of a DSM cell illustrating the stimulatory effect of 17β-estradiol (100 nM) on NPo in inside-out configuration. A portion of recording before and after application of 17β-estradiol (100 nM) is shown in expanded time scales, see FIGS. 4B and 4C). Summary data illustrating the stimulatory effects of 17β-estradiol (100 nM) on NPo, see FIG. 4B, and single BK channel current amplitude, FIG. 4C, observed in inside-out patches (n=9, N=7; *P<0.05). Post treatment of DSM cell membrane patches with paxilline (1 μM) completely abolishes the NPo of single BK channels (n=5, N=5; *P<0.05). ‘C’ and ‘O’ represent the closing and opening states of BK channels, respectively. However, 17β-estradiol (100 nM) did not affect the single BK channel current amplitude, which was 11.7±0.7 pA and 11.9±0.5 pA in the absence and presence of 17β-estradiol, respectively (n=9, N=7; P>0.05; FIG. 4). As illustrated in FIG. 4A the selective BK channel inhibitor paxilline (1 μM) completely abolished single BK channel activity (n=5, N=5; P<0.05), suggesting that the stimulatory effect of 17β-estradiol on channel NPo was due to BK channel activation. The results from these experiments support the concept that 17β-estradiol rapidly activates the BK channels by directly targeting the channel, rather than intracellular signaling pathways.

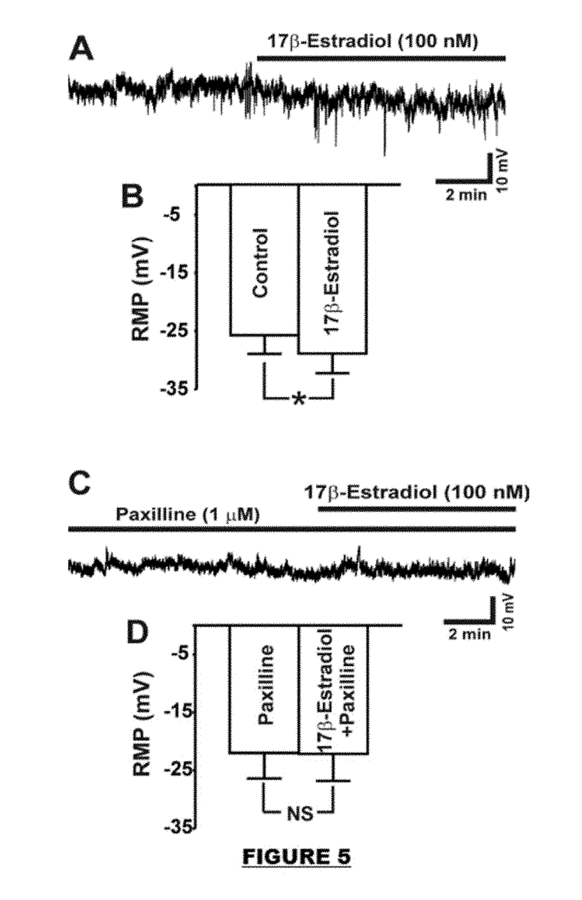

BK channel activation with 17β-estradiol hyperpolarizes the resting membrane potential (RMP) in DSM cells. Next, we aimed to investigate the BK channel-dependent regulation of the human DSM cell RMP by 17β-estradiol. The current-clamp experiments showed that 17β-estradiol (100 nM) significantly hyperpolarized the DSM cell RMP by ˜3 mV, from a control value of −25.7±3.2 mV to −28.9±3.3 mV in the presence of 17β-estradiol (n=11, N=9; P<0.05; FIG. 4A-B). As shown in FIG. 4C, 17β-estradiol had no effect on the human DSM cell RMP when administered in the presence of the BK channel inhibitor paxilline (1 μM), with the RMP of −21.9±4.4 mV in comparison to the control value (paxilline only) of −22.1±4.7 mV (n=8, N=5; P>0.05; FIG. 5D). These data support the concept that 17β-estradiol regulates the human DSM cell RMP through a BK channel-dependent mechanism.

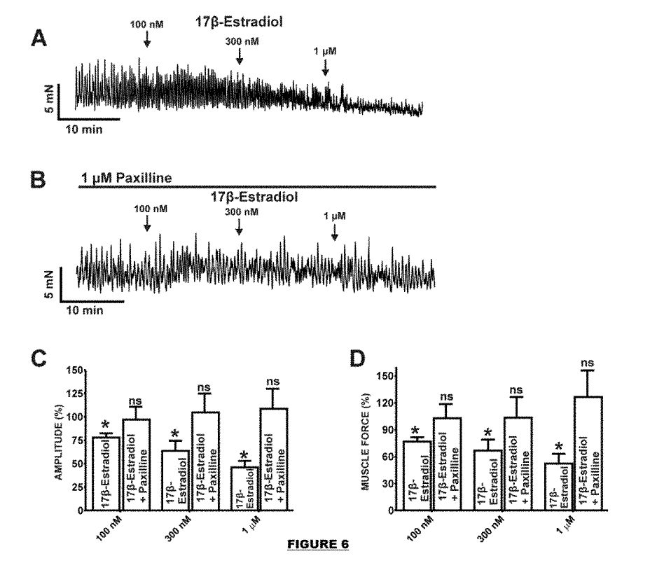

17β-Estradiol reduces spontaneous phasic contractions of human DSM isolated strips in a BK channel-dependent manner. To investigate the regulatory role of 17β-estradiol on human DSM contractility, we performed functional studies with human DSM isolated strips. 17β-Estradiol (100 nM-1 μM) significantly inhibited the spontaneous phasic contraction amplitude and muscle force integral (defined as area under the time-force curve) in a concentration-dependent manner (n=8, N=5; P<0.05; FIG. 6). 17β-Estradiol (1 μM) decreased human DSM spontaneous phasic contraction amplitude and muscle force integral by 53.3±8.9% and 44.4±13.2%, respectively (n=8, N=5; P<0.05; FIG. 6), without significantly changing phasic contraction frequency (n=8, N=5; P>0.05). The inhibitory effect of 17β-estradiol on human DSM contractility was abolished by 1 μM paxilline, a selective BK channel inhibitor. As shown in FIG. 6, in DSM strips pre-incubated with the selective BK channel inhibitor paxilline (1 μM), 17β-estradiol (100 nM-1 μM) did not affect spontaneous phasic contraction amplitude and muscle force integral (n=5, N=5; P>0.05, FIG. 6B-D). The data suggest that 17β-estradiol decreases spontaneous phasic contractions in human DSM, and this effect is mediated by BK channels.

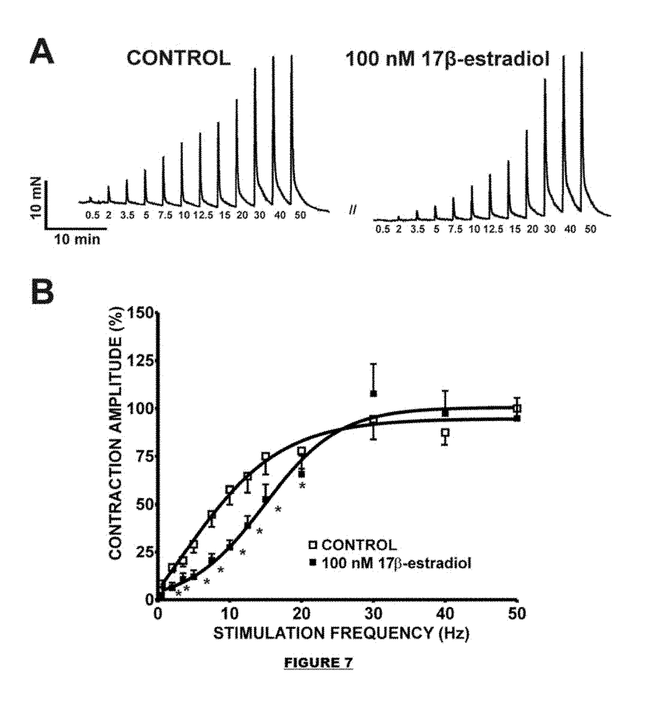

17β-Estradiol reduces nerve-evoked contractions of human DSM isolated strips. In the next series of isometric DSM tension recording experiments, we tested the effects of 17β-estradiol on nerve-evoked contractions induced by electrical field stimulation (EFS) at frequencies ranging 0.5-50.0 Hz. The EFS pulse parameters were as follows: 0.75-ms pulse width, 20-V pulse amplitude, 3-s stimulus duration. As shown in FIG. 7, 17β-estradiol (100 nM) significantly inhibited the EFS-induced contractions, in particular, at lower stimulation frequencies (0.5-20.0 Hz) (n=7, N=3; P<0.05).

Discussion

BK channels are key determinants of human DSM function and their activity is dually controlled by intracellular Ca²⁺ and voltage ENREF 3. Until now, the regulatory role of estrogens on BK channel activity has never been investigated in human DSM. Detailed knowledge about the regulation of DSM excitability and contractility by estrogens is particularly important in human DSM, since humans are the target species for therapeutic interventions. Substantial interspecies differences in the anatomy and physiology of the lower urinary tract are well documented. Therefore, the information obtained from experimental animal models cannot be directly translated to humans.

The current disclosure investigated the underlying molecular and cellular mechanism by which 17β-estradiol regulates human DSM function. We utilized a combined experimental approach using nanomolar concentrations (100 nM) of 17β-estradiol to examine its physiological role in regulating excitation-contraction coupling in human DSM. A key aspect of the current disclosure is that for the first time, the effects of 17β-estradiol on BK channel activation have been elucidated directly in clinically-characterized human DSM at both the cellular and tissue levels. We provided compelling novel evidence to support the concept that under physiological conditions, 17β-estradiol decreases human DSM excitability and contractility through a mechanism involving direct BK channel activation.

In non-DSM smooth muscle, such as bovine aortic, canine colonic, and human coronary artery smooth muscle cells, 17β-estradiol increased BK channel activity through a direct interaction with the regulatory β₁-subunit of the BK channel. In DSM, this mechanism could have an important physiological role, since the BK channel β₁-subunit is critical for DSM contractility. A recent study in guinea pig DSM is in support of this hypothesis, and further revealed that 17β-estradiol activates whole cell BK currents, TBKCs, and hyperpolarizes the DSM cell RMP via direct BK channel stimulation. Consistent with these findings, the current disclosure's data from whole cell patch-clamp recordings showed that 17β-estradiol increased depolarization induced steady-state whole-cell K⁺ currents in freshly-isolated DSM cells. These stimulatory effects of 17β-estradiol were abolished by the selective BK channel inhibitor paxilline, suggesting a mediatory role of the BK channels (FIG. 2).

It is important to consider that the underling cellular mechanism of 17β-estradiol-induced BK channel activation may include direct stimulation of the BK channel, or indirect pathways including modulation of intracellular Ca²⁺ dynamics that affect BK channel Ca²⁺ sensitivity. Single BK channel recordings from inside-out excised membrane-patches from guinea pig DSM cells have shown 17β-estradiol to significantly increase BK channel NPo, confirming a direct non-genomic mechanism of 17β-estradiol in the regulation of BK channel activity. In line with this recent study, The current disclosure's data from human DSM inside-out excised membrane patches revealed a significant increase in BK channel NPo by 17β-estradiol (FIG. 4A-B), without affecting the single BK channel amplitude (FIG. 4C). Two important conclusions can be drawn from these observations. First, the rapid stimulatory effects of 17β-estradiol on human DSM BK channel activity indicate that 17β-estradiol regulates BK channel activity through a mechanism independent of the genomic estrogen receptors. Second, the stimulatory effects of 17β-estradiol on BK channel NPo (FIG. 4A-B) are consistent with direct modulation of the smooth muscle-specific regulatory BK channel β₁-subunit by 17β-estradiol.

The single channel experiments (FIG. 4) could explain why 17β-estradiol increases the frequency of TBKCs in DSM cells, without significant effects on TBKC amplitude (FIG. 4). The increase in TBKC frequency could be attributed to an increase in BK channel NPo by 17β-estradiol (FIG. 4), which can thus increase the number of single BK channel opening events at −20 mV in DSM cells. Therefore, 17β-estradiol increased the number of small BK channel opening events reaching the TBKC threshold of 9 pA as a result of direct modulation of BK channel NPo, while having no potentiating effects on the average amplitude of TBKCs in DSM cells.

The BK channel β₁-subunit, known to be highly expressed in human DSM, is involved in regulation of DSM contractility. Therefore, modulation of BK channel activity via the β₁-subunit may have clinical relevance in the treatment of OAB. 17β-Estradiol can modulate BK channel activity by binding to the regulatory BK channel β₁-subunit causing direct channel activation. The current disclosure has provided for the first time evidence that 17β-estradiol inhibits human DSM cell excitability and contractility by direct BK channel activation. This cellular mechanism should be considered when novel hormonal therapies for bladder dysfunction are being developed.

On the other hand, the current disclosure cannot completely exclude alternative contributing mechanisms including 17β-estradiol-induced perturbation of the membrane environment surrounding the BK channel. Many different types of ion channels, including the BK channel, are known to be modulated by fatty acids, lipids, and other membrane active agents. In addition, estrogens can also potentially affect BK channel expression and splice variants indirectly by genomic mechanisms. Whether these alternative mechanisms operate in parallel to precisely control excitability and contractility in human DSM reminds to be investigated.

It is well known that the membrane potential of human DSM cell is actively regulated by the BK channels. Inhibition of the BK channels leads to DSM cell membrane depolarization, while pharmacological activation of the BK channel with selective channel openers results in DSM cell membrane hyperpolarization. Similarly, in rodent DSM cells 17β-estradiol hyperpolarizes DSM cell membrane. In the current disclosure, for the first time in human DSM cells, we demonstrated that activation of BK channels with 17β-estradiol significantly hyperpolarized the RMP in a BK-channel-dependent manner (FIG. 5). These results are consistent with previous findings in guinea pig DSM, and clearly demonstrate a regulatory role for 17β-estradiol on the human DSM cell RMP. FIG. 4 shows at FIG. 5A a representative trace of an RMP recording in current-clamp mode illustrating the hyperpolarizing effects of 17β-estradiol (100 nM) in an isolated human DSM cell. FIG. FIG. 5B shows summary data illustrating the hyperpolarizing effects of 17β-estradiol on the human DSM cell RMP (n=11, N=9; *P<0.05). FIG. 5C illustrates a representative trace of an RMP in current-clamp mode demonstrating that when the BK channels are blocked with 1 μM paxilline, 17β-estradiol (100 nM) did not cause membrane hyperpolarization in DSM cells. FIG. 5D shows a summary data illustrating that 17β-estradiol had no effect on the human DSM cell RMP in the presence of 1 μM paxilline (n=8, N=5; P>0.05).

DSM cell membrane hyperpolarization attenuates L-type voltage-gated Ca²⁺ channel activity, decreases intracellular Ca²⁺ concentrations, thus causing DSM relaxation. The current disclosures data shows that under physiological condition estrogens are essential modulators of the human DSM RMP (FIG. 5), which in turn affects DSM contractility. Indeed, the current disclosure's functional studies of DSM contractility showed that 17β-estradiol significantly inhibited the amplitude and force of spontaneous phasic contractions (FIG. 6) in human DSM isolated strips. Paxilline, a selective BK channel blocker, prevented the relaxant effect of 17β-estradiol on spontaneous phasic contractility suggesting that 17β-estradiol inhibits human DSM contractions primarily by its stimulatory effects on BK channel activity (FIG. 6). FIG. 6 shows at 6A and 6B representative original recordings illustrating the concentration-dependent inhibitory effects of 17β-estradiol (100 nM-1 μM) on spontaneous phasic contractions of isolated DSM strips in the absence (FIG. 6A) or presence (FIG. 6B) of paxilline (1 μM). FIGS. 6C and 6D illustrate summary data illustrating the inhibitory effects of 17β-estradiol on the amplitudes and muscle force of spontaneous phasic contraction of DSM isolated strips in the absence (n=8, N=5) and presence of 1 μM paxilline (n=5, N=5; *P<0.05).

The current disclosure designed the EFS experiments to assess the effects of 17β-estradiol on nerve-evoked contractions over a large range of stimulation frequencies (0.5-50.0 Hz). The results showed that in human DSM, 17β-estradiol reduced EFS-induced contractions, particularly at the lower and more physiologically-relevant stimulation frequencies (FIG. 7), indicating that 17β-estradiol regulates nerve-evoked human DSM contractions. Therefore, targeting the BK channels with 17β-estradiol could represent a novel pharmacological approach to control neurogenically mediated detrusor dysfunction in female patients. FIG. 7 shows at FIGS. 7A and 7B representative original traces illustrating the inhibitory effect of 17β-estradiol (100 nM) on the EFS-induced contraction of DSM isolated strips. FIG. 7C illustrates summary data illustrating the inhibitory effects of 17β-estradiol on the amplitude of the EFS-induced contractions of DSM isolated strips (n=7, N=3; *P<0.05). The data were normalized to contraction amplitude at the stimulation frequency of 50 Hz (prior to 17β-estradiol addition) taken as 100% and were presented as percentages (%).

It has been shown that high, non-physiological concentrations of 17β-estradiol (30 μM), cause a decrease in the pharmacologically-induced and nerve-evoked contractions in rat DSM isolated strips. In another study, 17β-estradiol (30 μM) induced inhibitory effects on the KCl- and muscarinic receptor-induced contractions of pig DSM isolated strips. Estrogen receptor antagonists did not affect the 17β-estradiol-induced inhibitory effects on the pharmacologically-induced DSM contractions suggesting no functional role for the estrogen receptors in mediating 17β-estradiol inhibitory effects on DSM contractions.

In the current disclosure, it has been demonstrated that 17β-estradiol decreases both spontaneous and nerve-evoked human DSM contractions at nanomolar concentrations (FIGS. 6 and 7). These concentrations are significantly below that of earlier reports that used non-physiological micromolar concentrations of 17β-estradiol. Therefore, the current disclosure's data supports the concept that 17β-estradiol may regulate human DSM contractility under physiological conditions.

In conclusion, for the first time directly in human DSM, the current disclosure's results reveal that 17β-estradiol regulates DSM excitability and contractility in a BK-channel-dependent manner. The study provides compelling evidence that 17β-estradiol exhibited direct non-genomic stimulatory effects on BK channels, thus decreasing the excitability and contractility of human DSM. The combined results support the idea that activation of BK channels with estrogens may represent a novel and effective treatment for patients with OAB and associated detrusor overactivity.

The BK (Big Potassium; large conductance Ca2+-activated K+ channel) channel is a major ion channel in the smooth muscle of the urinary bladder wall. BK channels, also called Maxi-K or slo1, are potassium channels characterized by their large conductance for potassium ions (K+) through cell membranes. These channels are activated (opened) by changes in membrane electrical potential and/or by increases in concentration of intracellular calcium ion (Ca2+). Opening of BK channels allows K+ to passively flow through the channel, down the electrochemical gradient. Under typical physiological conditions, this results in an efflux of K+ from the cell, which leads to cell membrane hyperpolarization (an increase in the electrical potential across the cell membrane) and a decrease in cell excitability (a decrease in the probability that the cell will transmit an action potential).

BK channels are essential for the regulation of several key physiological processes including smooth muscle tone and neuronal excitability. They control the contraction and relaxation of urinary bladder smooth muscle.

Selective pharmacological targeting of BK channels with estrogen (17beta-estradiol) may be a very effective novel approach to treat overactive bladder syndrome and other urological diseases. The challenge of BK channels being expressed in other tissues is not unique to therapeutic targets affecting lower urinary tract function, and fortunately this obstacle has been pharmacologically overcome in a number of disease states.

Considering the relatively higher BK channel expression in the urinary bladder compared to other tissues, as well as their important functional roles in urinary bladder, suggests that pharmacological manipulation of BK channels with estrogens in overactive bladder patients may provide effective treatment, with minimal adverse collateral cardiovascular effects. The potential clinical application of BK channel modulation with estrogens for overactive therapy should be considered as a novel approach for overactive bladder management, and further validated in clinical trials.

Detrusor smooth muscle (DSM) cells represent the primary functional unit of the urinary bladder and their dysregulation is responsible for a significant portion of lower urinary tract dysfunction. Ion channels expressed in DSM control urinary bladder function, and therefore they represent promising alternative targets for the pharmacological intervention of OAB. Selective manipulation of individual ion channel subtypes in DSM could substantially alleviate various types of bladder dysfunction such as OAB, urinary incontinence, or detrusor underactivity while potentially minimizing collateral adverse effects elsewhere in the body. The current disclosure relates to selective BK channel targeting with estrogens and is a novel pharmacological approach to control OAB.

Estrogens have an essential role in regulating detrusor smooth muscle (DSM) function. However, the underlying molecular and cellular mechanisms by which estrogens control human DSM excitability and contractility are not well known.

Human DSM specimens from open bladder surgeries on 24 patients were used to elucidate the mechanism by which 17β-estradiol regulates large conductance voltage- and Ca2+-activated K+ (BK) channels, the most prominent K+ channels in human DSM. The perforated patch-clamp technique, as known to those of skill in the art, was used on freshly-isolated DSM cells and DSM tension recordings to investigate the mechanism by which 17β-estradiol activates BK channels.

17β-Estradiol (100 nM) rapidly increased depolarization-induced whole cell K+ currents in human DSM cells. This 17β-estradiol-induced potentiation of the whole cell BK currents was completely abolished by the selective BK channel inhibitor paxilline (1 μM), clearly indicating that 17β-estradiol specifically activates BK channels. 17β-Estradiol also increased the frequency of ryanodine receptor-mediated transient BK currents. Single BK channel recordings showed that 17β-estradiol (100 nM) significantly increased the BK channel open probability of inside-out excised membrane patches, revealing that 17β-estradiol activates BK channels directly. 17β-Estradiol reduced spontaneous phasic contractions of human DSM isolated strips in a concentration-dependent manner (100 nM-1 μM). The inhibitory effects of 17β-estradiol on DSM spontaneous phasic contractions were blocked by paxilline (1 μM). 17β-Estradiol (100 nM) also reduced nerve-evoked contractions of human DSM strips.

Collectively, the results reveal that 17β-estradiol plays a critical role in regulating human DSM function through a direct non-genomic activation of BK channels. The combined results support the concept that activation of BK channels with estrogens may represent a novel and effective treatment for patients with overactive bladder (OAB) and associated detrusor overactivity.

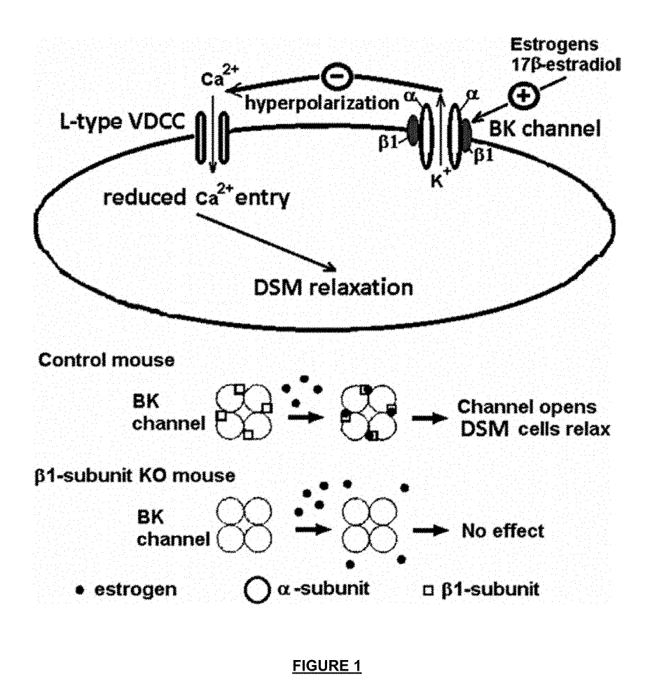

FIG. 1 shows a proposed mechanism of estrogen effects on BK channels in DSM cells. Estrogens activate the BK channels in DSM cells by directly binding to the channel, presumably to the regulatory b1-subunit. This causes membrane potential hyperpolarization, closure of the L-type VDCC, and subsequent DSM relaxation. Theoretically, estrogens should activate BK channels only in the presence of the regulatory b1-subunit and not in DSM from b1-subunit knockout mouse (KO).

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A method of regulating detrusor smooth muscle function comprising:
administering a compound comprising an estrogen hormone;
regulating excitation-contraction coupling in detrusor smooth muscle via BK channel activation;
increasing depolarization of detrusor smooth muscle cells; and
reducing EFS-induced contractions at physiologically-relevant stimulation frequencies.

2. The method of claim 1, wherein the estrogen hormone comprises 17β-estradiol.

3. The method claim 1, wherein a potassium channel blacker is administered to counteract stimulatory effects of the estrogen hormone.

4. The method of claim herein the estrogen hormone increases BK channel NPo.

5. The method of claim 4, wherein BK channel amplitude remains substantially unaffected.

6. The method of claim 1, wherein regulating detrusor smooth muscle function is achieved independent of genomic estrogen receptors.

7. The method of claim 1, wherein regulating detrusor smooth muscle function is achieved by direct modulation of the smooth muscle-specific regulatory BK channel β₁-subunit.

8. The method of claim 1, wherein the estrogen hormone hyperpolarizes a resting membrane potential in a BK-channel-dependent manner.

9. The method of claim 1, wherein the physiologically-relevant stimulation frequencies are from 0.5 to 50.0 Hz.

10. A hormonal therapeutic intervention comprising:
administering a therapeutic agent comprising an estrogen hormone;
regulating excitation-contraction coupling in detrusor smooth muscle via BK channel activation; and
controlling neurogenically mediated detrusor dysfunction.

11. The method of claim 10, wherein the estrogen hormone comprises 17β-estradiol.

12. The method of claim 11, wherein 17β-estradiol rapidly activates the BK channels by directly targeting the BK channel rather than targeting intracellular signaling pathways.

13. The method claim 10, wherein a potassium channel blocker is administered to counteract stimulatory effects of the estrogen hormone.

14. The method of claim 10, wherein the estrogen hormone increases BK channel NPo.

15. The method of claim 14, wherein BK channel amplitude remains substantially unaffected.

16. The method of claim 10, wherein regulating detrusor smooth muscle function is achieved independent of genomic estrogen receptors.

17. The method of claim 10, wherein regulating detrusor smooth muscle function is achieved by direct modulation of a smooth muscle-specific regulatory BK channel β₁-subunit.

18. The method of claim 10, wherein the estrogen hormone hyperpolarizes a resting membrane potential in a BK-channel-dependent manner.
